(19) United States
(12) Patent Application Publication
(10) Pub. No.: US 2026/0248826 A1
(43) Pub. Date: Aug. 27, 2026

(54) DRUG COMBINATION FOR TREATMENT OF PANCREATIC CANCER

(71) Applicant: MINNEAMRITA THERAPEUTICS LLC, Tampa, FL (US)

(72) Inventors: Mohana R. VELAGAPUDI, Tampa, FL (US); Ashok Kumar SALUJA, Tampa, FL (US)

(73) Assignee: MINNEAMRITA THERAPEUTICS LLC, Tampa, FL (US)

(21) Appl. No.: 19/163,760

(22) PCT Filed: Mar. 21, 2023
(86) PCT No.: PCT/US2023/015813

§ 371 Date: Sep. 9, 2025

Related U.S. Application Data
(60) Provisional application No. 63/489,221, filed on Mar. 9, 2023.

(51) Int. Cl. A61K 31/665; A61K 31/337; A61K 31/365; A61K 31/7068; A61P 35/00

(52) U.S. Cl. CPC A61K 31/665; A61K 31/337; A61K 31/365; A61K 31/7068; A61P 35/00

(57) ABSTRACT

The cancer agents minnelide, nab-paclitaxel, and gemcitabine were used in combination for the synergistic treatment of pancreatic cancer in cancer subjects. Cancer subjects treated daily with the combination had experienced side effects. Therefore, described herein is a dosing regimen for the combination that reduces the number of side effects experienced by cancer subjects. The dosing regimen was timed on a 28-day cycle where minnelide was dosed once per day on days 1 to 5, 8 to 12, and 15 to 19 of the cycle, and nab-paclitaxel and gemcitabine was dosed once per day on days 1, 8, and 15 of the cycle. The described dosing regimen has been shown to be well tolerated in clinical trials.

### RELATED APPLICATIONS

This application claims priority under 35 U.S.C. § 119 (e) to U.S. Provisional Patent Application No. 63/489,221 filed Mar. 9, 2023, which is incorporated herein by reference.

### BACKGROUND OF THE INVENTION

Minnelide™ is a prodrug that rapidly releases the active compound triptolide when exposed to phosphatases in the bloodstream. Triptolide is a diterpene that has been shown to inhibit tumor cell proliferation and induce apoptosis in vitro and in animal models of cancer, including human mammary tumors (Shamon et al., Cancer Lett. 1997; 112:113-7), cholangio-carcinoma cells (Tengchaisri et al., Cancer Lett. 1998; 133:169-75), xenografts of several different tumor types, including melanoma, breast cancer, bladder cancer, gastric carcinoma (Yang et al., Mol Cancer Ther. 2003; 2:65-72), pancreatic tumors (Phillips et al., Cancer Res. 2007; 67 (19): 9407-16) and neuroblastoma (Antonoff et al., Surgery. 2009; 146:282-90).

The antitumor effect of triptolide is the result of inhibition of heat shock protein (HSP) 70 expression in tumor cells and induction of apoptosis. While the mechanism of action of triptolide inhibition of HSP70 expression has not been fully elucidated, it has been shown to induce caspase activation (Choi et al., Biochem Pharmacol. 2003; 66:273-80; Liu et al., Biochem Biophys Res Commun. 2004; 319:980-6; Wang et al., J Mol Med. 2006; 84:405-15; Carter et al., Blood. 2006; 108:630-7).

In the MIA-PaCa2 and PANC-1 pancreatic cancer cell lines, cytochrome c release from mitochondria into the cytosol and caspase-3 activity were significantly increased after incubation with triptolide (TPL), suggesting that the mitochondrial apoptotic pathway is responsible for TPL-induced cell death in these cells (Phillips et al., Cancer Res. 2007; 67 (19): 9407-16). Further studies showed that inhibition of HSP70 in the pancreatic tumors by TPL was mediated by inhibition of transcriptional activity of specificity protein 1 (Sp1) by TPL (Banerjee et al., Biol Chem. 2013 Nov. 22;288 (47)).

Sp1 is a transcription factor that regulates a number of pro-survival pathways like nuclear factor kappa-light-chain-enhancer of activated B cells (NF-kB) and Heat shock factor 1 (HSF1). Inhibition of Sp1 transcriptional ability downregulates NF-kB activity thereby increasing apoptosis. Further, studies showed that activation of HSF1 (the key transcription factor for HSP70) was also regulated via Sp1 and NF-kB activity.

Along with affecting the tumor cells, minnelide also depleted the extracellular matrix (ECM) of the pancreatic tumors by downregulating hyaluronan (HA) synthesis and preventing collagen cross linking. This in turn resulted in better vasculature and better drug delivery to the tumor.

In addition, TPL induces autophagic cell death in some tumor types: the metastatic lines S2-013, S2-VP10 and Hs766T. TPL-induced autophagy requires autophagy-specific genes (atg5 or beclin1) and is associated with inactivation of the protein kinase B (Akt)/mammalian target of rapamycin/p70S6K pathway and up-regulation of the extracellular signal-related kinase (ERK) ½ pathway.

Studies showed that at a dose of 0.2 mg/kg, minnelide disrupts transforming growth factor-β (TGFβ) signaling in cancer associated fibroblasts (CAFs) and prevents tumor-stroma crosstalk, leading to decreased oncogenic signaling in the tumor epithelial cells.

The tumor microenvironment in pancreatic ductal adenocarcinoma is highly heterogeneous, fibrotic, and hypovascular, marked by extensive desmoplasia and maintained by the tumor cells, CAFs and other stromal cells. A growing number of studies implicate the role of regulatory DNA elements called super-enhancers (SE) in maintaining cell-type-specific gene expression networks in both normal and cancer cells.

Using chromatin activation marks, Noel et al. (Oncogenesis. 2020 Nov. 9; 9 (11): 100) first mapped SE networks in pancreatic CAFs and epithelial tumor cells and found them to have distinct SE profiles. Next, they explored the role of TPL, a natural compound with antitumor activity, in the context of modulating cell-type-specific SE signatures in pancreatic ductal adenocarcinoma (PDAC). They found that TPL, cytotoxic to both pancreatic tumor cells and CAFs, disrupted SEs in a manner that resulted in the downregulation of SE-associated genes (e.g., BRD4, MYC, RNA Pol II, and Collagen 1) in both cell types at mRNA and protein levels. Their observations suggest that TPL acts as an SE interactive agent and may elicit its antitumor activity through SE disruption to reprogram cellular cross talk and signaling in PDAC.

Based on their findings, epigenetic reprogramming of transcriptional regulation using SE modulating compounds such as TPL may provide means for effective treatment options for pancreatic cancer patients.

Despite the availability of antitumor agents, there is an urgent need to develop treatment strategies that not only target the tumor cells but can also modulate the stromal cells. Thus, novel therapeutic agents and treatment regimens that are better tolerated and more effective are desirable.

### SUMMARY

Minnelide™ (structure shown below) was determined to have potent antitumor activity against pancreatic and ovarian tumor cells in vitro (when “activated” with alkaline phosphatase to release triptolide) and markedly improved animal survival and significantly reduced tumor volumes and weights when administered daily in models of pancreatic and ovarian cancer.

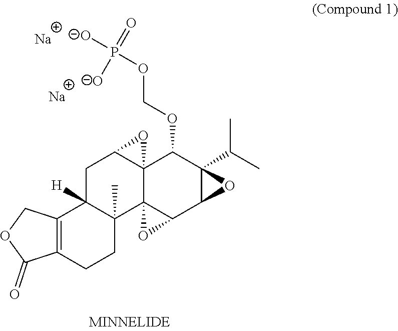

Accordingly, this disclosure provides a method for treating pancreatic cancer in a cancer subject, the method which comprises administering to the cancer patient during a 28 day cycle a therapeutically effective combination of:
a) about 0.25 mg to about 2.0 mg of minnelide according to a first regimen wherein a dose is given once per day on days 1 to 5, 8 to 12, and 15 to 19 of the cycle;
b) about 75 mg/m² to about 125 mg/m² of nab-paclitaxel according to a second regimen wherein a dose is given once per day on days 1, 8, and 15 of the cycle; and
c) about 600 mg/m² to about 1000 mg/m² of gemcitabine according to the second regimen of the cycle;

wherein the cycle is repeated one or more times and the combination effectively treats the pancreatic cancer.

The described technology provides for the use of the compositions described herein for use in medical therapy. The medical therapy can be treating cancer, for example, breast cancer, gastric cancer, lung cancer, pancreatic cancer, prostate cancer, or colon cancer. The invention also provides for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-B. Mean tumor volumes (A) and tumor weights (B) 28 days after initiation of once daily po treatment with gemcitabine/abraxane with or without minnelide in an orthotopic S2-VP10 pancreatic tumor model.

### DETAILED DESCRIPTION

A preclinical study of minnelide was conducted in mice. Female athymic nude mice (NCI nu/nu; 4 to 6 weeks old) were anesthetized with xylazine/ketamine, and S2-VP10 tumor cells embedded in Matrigel were implanted into the pancreatic tail (3×105 cells/mouse). Twelve days after tumor implantation (Day 0), animals were randomized to receive saline, nab-paclitaxel plus gemcitabine (Gem/Abx; 250/25 mg/kg), or minnelide plus nab-paclitaxel plus gemcitabine (Minn/Gem/Abx; 0.15/250/25 mg/kg or 0.21/250/25 mg/kg) (10 animals/group). Minnelide was administered once daily (QD) by oral gavage (PO). Treatment continued until Day 28 when animals were sacrificed.

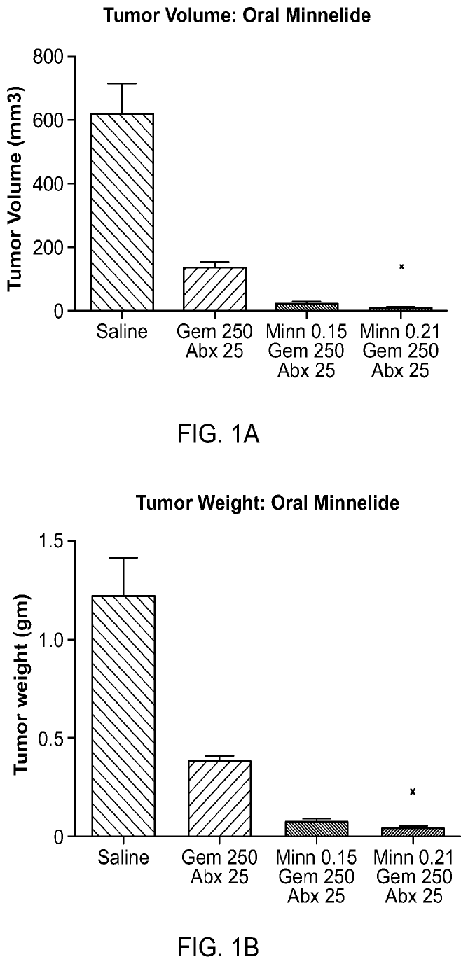

Treatment with Gem/Abx resulted in reduced tumor volumes and weights on Day 28 compared with saline-treated (vehicle) controls. The addition of minnelide at a dose of 0.15 mg/kg or 0.21 mg/kg further reduced tumor volumes and weights compared with Gem/Abx treatment alone (FIG. 1).

Overall, daily oral treatment with minnelide decreased tumor volumes and weights when administered concurrently with Gem/Abx treatment compared with minnelide or Gem/Abx treatment alone in the orthotopic S2-VP10 pancreatic tumor model.

Additional information and data supporting the invention can be found in U.S. Provisional Patent Application No. 63/489,205, filed by the inventors on Mar. 9, 2023, and U.S. Pat. Nos. 9,150,600 and 8,507,552, which application and patents are incorporated herein by reference in their entirety.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of +5%, +10%, +20%, or +25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, the patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

The term “adverse event” refers to any untoward medical occurrence associated with the use of a drug in humans, whether or not considered drug related.

The term “serious adverse event” refers to death, a life-threatening adverse event, inpatient hospitalization or prolongation of existing hospitalization, a persistent or significant incapacity or substantial disruption of the ability to conduct normal life functions, or a congenital anomaly/birth defect. Important medical events that may not result in death, be life-threatening, or require hospitalization may be considered serious when, such medical events include allergic bronchospasm requiring intensive treatment in an emergency room or at home, blood dyscrasias or convulsions that do not result in inpatient hospitalization, or the development of drug dependency or drug abuse, etc.

The term “regimen” refers to a course of medical treatment over a period of time. The regimen described herein includes one or more sub-regimens, such as a first regimen, a second regime, and so on, that make up the entire regimen of medical treatment. The period of time for treatment may be one or more days, one or more weeks, one or more months, etc., wherein the period of time may be repeated in a cyclic fashion one or more times. For example, a period of time may be 28 days, defining the length of a cycle that may be repeated. A regimen may be a 28 day cycle where a compound A is administered on specific days of the cycle, defining a first regimen, and where compound B is administered on other specific days of the cycle that define a second regimen, which in total defines the entire regimen.

The IUPAC chemical name for the compound MINNELIDE or Minnelide™, the drug, is: disodium; [(1S,2S,4S,5S,7S,8R,9R,11S,13S)-1-methyl-17-oxo-7-propan-2-yl-3,6,10,16-tetraoxaheptacyclo[11.7.0.0^{2,4}.0^{2,9}.0^{5.7}.09^{,11}.0^{14,18}] icos-14 (18)-en-8-yl]oxymethyl phosphate. Herein, MINNELIDE and other pharmaceutically acceptable salt forms thereof will be referred to as compound 1.

### Classification of Adverse Events by Severity.

The severity of each Adverse Event (AE) follows guidelines from the National Cancer Institute Common Terminology Criteria for Adverse Events (NCI CTCAE). A brief summary is provided below:

Grade 1: An AE that is transient or mild discomfort, not interfering with the patient's daily activity performance or functioning; medical intervention/therapy may be required.

Grade 2: An AE of sufficient severity as to possibly make the patient moderately uncomfortable; possibly influencing the patient's daily activity performance or functioning; generally, not impairing the patient's ability to continue in the study; and/or possibly needing therapeutic intervention.

Grade 3: An AE event generally causing severe discomfort, significantly influencing the patient's daily activity performance or functioning, generally requiring alteration or cessation of study drug administration, and/or generally requiring therapeutic intervention with hospitalization possible.

Grade 4: An AE that is considered to be life threatening, resulting in significant disability or incapacity, and/or representing the worst possible occurrence of that event with hospitalization probable.

Grade 5: A death related to an AE.

### Embodiments of the Technology

This disclosure provides a method for treating cancer in a cancer subject, the method which comprises administering to the cancer patient during a 28 day cycle a therapeutically effective combination of:
a) about 0.25 mg to about 2.0 mg of minnelide according to a first regimen wherein a dose is given once per day on days 1 to 5, 8 to 12, and 15 to 19 of the cycle;
b) about 75 mg/m² to about 125 mg/m² of nab-paclitaxel according to a second regimen wherein a dose is given once per day on days 1, 8, and 15 of the cycle; and
c) about 600 mg/m² to about 1000 mg/m² of gemcitabine according to the second regimen of the cycle;

wherein the cycle is repeated one or more times and the combination effectively treats the cancer.

In various embodiments, the cancer subject is administered about 0.25 mg to about 1.00 mg of minnelide according to the first regimen. In various embodiments, according to the first regimen, the cancer subject is administered a milligram amount of minnelide that is about 0.15 mg, 0.25 mg, 0.35 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.2 mg, 2.3 mg, 2.4 mg, 2.5 mg, 2.6 mg, 2.7 mg, 2.8 mg, 2.9 mg, 3.0 mg, 3.5 mg, 4.0 mg, 4.5 mg, 5.0 mg, 5.5 mg, or about 6.0 mg.

In various embodiments, the cancer subject is administered about 50 mg/m² to about 100 mg/m² of paclitaxel according to the second regimen. In various embodiments, according to the second regimen, the cancer subject is administered a milligram of paclitaxel that is about 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 125 mg/m², 150 mg/m², 175 mg/m², or about 200 mg/m².

In some embodiments, the cancer subject is administered about 0.25 mg of minnelide according to the first regimen, about 75 mg/m² of nab-paclitaxel and about 600 mg/m² of gemcitabine each according to the second regimen.

In some embodiments, the cancer subject is administered about 0.25 mg of minnelide according to the first regimen, about 100 mg/m² of nab-paclitaxel and about 800 mg/m² of gemcitabine each according to the second regimen.

In some embodiments, the cancer subject is administered about 0.25 mg of minnelide according to the first regimen, about 125 mg/m² of nab-paclitaxel and about 1000 mg/m² of gemcitabine each according to the second regimen.

In some embodiments, the cancer subject is administered about 0.50 mg of minnelide according to the first regimen, about 125 mg/m² of nab-paclitaxel and about 1000 mg/m² of gemcitabine each according to the second regimen.

In some embodiments, the cancer subject is administered about 0.75 mg of minnelide according to the first regimen, about 125 mg/m² of nab-paclitaxel and about 1000 mg/m² of gemcitabine each according to the second regimen.

In some embodiments, the cancer subject is administered about 1.00 mg of minnelide according to the first regimen, about 125 mg/m² of nab-paclitaxel and about 1000 mg/m² of gemcitabine each according to the second regimen.

In some embodiments, the cancer subject is administered about 1.25 mg of minnelide according to the first regimen, about 125 mg/m² of nab-paclitaxel and about 1000 mg/m² of gemcitabine each according to the second regimen.

In some embodiments, the cancer subject is administered about 1.50 mg of minnelide according to the first regimen, about 125 mg/m² of nab-paclitaxel and about 1000 mg/m² of gemcitabine each according to the second regimen.

In some embodiments, the cancer subject is administered about 1.75 mg of minnelide according to the first regimen, about 125 mg/m² of nab-paclitaxel and about 1000 mg/m² of gemcitabine each according to the second regimen.

In some embodiments, the cancer subject is administered about 2.00 mg of minnelide according to the first regimen, about 125 mg/m² of nab-paclitaxel and about 1000 mg/m² of gemcitabine each according to the second regimen.

In some embodiments, the cancer subject is administered about 1.25 mg of minnelide according to the first regimen, about 125 mg/m² of nab-paclitaxel and about 1000 mg/m² of gemcitabine each according to the second regimen.

In some embodiments, the cancer subject is administered about 1.50 mg of minnelide according to the first regimen, about 125 mg/m² of nab-paclitaxel and about 1000 mg/m² of gemcitabine each according to the second regimen.

In some embodiments, the cancer subject is administered about 1.75 mg of minnelide according to the first regimen, about 125 mg/m² of nab-paclitaxel and about 1000 mg/m² of gemcitabine each according to the second regimen.

In some embodiments, the cancer subject is administered about 2.00 mg of minnelide according to the first regimen, about 125 mg/m² of nab-paclitaxel and about 1000 mg/m² of gemcitabine each according to the second regimen.

In some embodiments, minnelide is administered orally or. In some embodiments, minnelide is administered intravenously. In various embodiments, nab-paclitaxel is administered intravenously or orally. In various embodiments, gemcitabine is administered intravenously or orally. In various embodiments, nab-paclitaxel is administered intravenously, concurrently or sequentially with gemcitabine. In various embodiments, gemcitabine is administered intravenously, concurrently or sequentially, with nab-paclitaxel.

In various embodiments, the cancer is gastric cancer, pancreatic cancer, ovarian cancer, breast cancer, bladder cancer, skin cancer, or a combination thereof. In some embodiments, cancer is pancreatic cancer. In some embodiments, the method effectively treats a cancer subject suffering from or further suffering from gastric cancer, pancreatic cancer, ovarian cancer, breast cancer, bladder cancer, skin cancer, or a combination thereof.

In various embodiments, the cancer subject is a human. In various embodiments, the cancer subject is a female human or a male human.

In various embodiments, the combination effectively treats the cancer without causing a complete blood count of the cancer subject to lower by more than 25%, 20%, 15%, 10%, or 5% from baseline. In various embodiments, the combination effectively treats the cancer without causing a platelet count or an absolute neutrophil count in the cancer subject to lower by more than 25%, 20%, 15%, 10%, or 5% from baseline.

In some embodiments, the methods described herein are used to treat a cancer that has become refractory, or to treat a cancer that is resistant or non-responsive to other methods of cancer treatment. In some embodiments, the pancreatic cancer is a cancer that has become refractory, resistant or non-responsive to other methods of cancer treatment. In some embodiments, the pancreatic cancer is metastatic adenocarcinoma of the pancreas.

### Clinical Experience.

Intravenous Minnelide. Phase 1, Multi-Center, Open-Label, Dose-Escalation, Safety, Pharmacokinetic, and Pharmacodynamic Study of minnelide given intravenously daily for 21 days followed by 7 days off schedule in patients with Advanced GI Tumors. A second dosing schedule of minnelide given intravenously QD for 5 days with 2 days off for three weeks followed by a 7-day rest period was also explored during this study. A total of 45 patients enrolled in the Phase 1 study, however 3 patients were not dosed with study drug. The remaining 42 patients were dosed in the study. The primary reason for the discontinuation of treatment was disease progression. There were 6 deaths in the study, but these occurred after the study drug had ended and were within the 30-day follow-up period. The deaths that occurred during the study were found to be not related to the study drug and were found to be related to progression of the patient's disease (pancreatic/gastric cancer). One patient who died from respiratory failure, and this was also found to be not related to use of the study drug.

There were 3 patients who discontinued the study drug due to a treatment emergent adverse events (TEAEs). All instances were considered Grade 3, and two of these TEAEs were considered to be probably related to use of the study drug. These two cases were also considered DLTs. A total of 28 Serious Adverse Events occurred in 17 patients and six were found to be related to the study drug.

Overall, the most commonly reported TEAE (>20%) regardless of causality included: hypoalbuminemia, anemia, hypoproteinemia, fatigue, neutropenia, leukopenia, thrombocytopenia, nausea, hypocalcemia, diarrhea, hyperphosphatemia, constipation, vomiting, hyponatremia, lymphopenia, abdominal pain, dehydration, hyperglycemia, peripheral edema, hypophosphatemia, and headache.

A total of 32 patients had at least 1 AE that was considered related to the study drug. Adverse events of neutropenia grade 3 and 4 of short durations have been observed at all dose levels and were determined to be drug related. The neutropenia resolved within a couple of days of not receiving treatment. Neutropenic fever and neutropenic infection were observed in three patients and determined to be drug related. Anemia and thrombocytopenia Grade 3 and 4 were observed in 22% of the patients and determined to be drug related. Other side effects of any grade that were possibly related to the drug included nausea, vomiting, diarrhea, constipation, anorexia, stomatitis, cerebellar toxicity, embolism and dyspnea, which occurred in ≤3% of the patients.

Oral Minnelide. A Phase 1, Multi-Center, Open-Label, Dose-Escalation, Safety, Pharmacokinetic, and Pharmacodynamic Study of Minnelide™ Capsules given alone or in combination with protein-bound paclitaxel in patients with Advanced Solid Tumors.

A total of 51 patients have been enrolled and dosed in the Phase 1 study, of whom 44 were evaluable for safety (34 in the monotherapy regimen and 16 in the combination regimen). Twenty-nine patients have completed treatment in the monotherapy regimen, and 6 dose escalations have occurred. In the monotherapy regimen, one DLT has been observed, which occurred in the 1.25 mg cohort. This patient did not complete full Cycle 1 dosing and later died due to Grade 5 related sepsis. As a result of this DLT, and out of an abundance of caution, 3 additional patients were enrolled in the 1.0 mg cohort.

A total of 45 SAEs have occurred in 25 patients and 8 were found to be related to the investigational product (IP) as follows: 1 event each of Grade 5 sepsis, Grade 4 sepsis, Grade 3 nausea, Grade 3 polymicrobial bacteraemia, Grade 3 intractable nausea, Grade 3 hypokalaemia, and Grade 2 enteritis in patients on monotherapy; one event each of Grade 5 sepsis and Grade 2 esophagitis in patients on combination therapy.

A total of 17 patients had been enrolled in combination therapy regimen. Two DLTs have been observed: one at the 0.25 mg minnelide and 125 mg/m² protein-bound paclitaxel dose level and one at the 0.25 mg minnelide and 100 mg/m² protein-bound paclitaxel dose level. One patient did not complete full cycle 1 dosing and was admitted to the hospital with unrelated Influenza A. The patient had also presented a Grade 4 decrease in lymphocytes possibly related to the IP. A total of 2 SAEs occurred in this patient, neither of which were deemed related to the IP. As this was the first patient in the combination regimen to receive protein-bound paclitaxel with minnelide, the combination regimen was put on enrolment hold until further escalation parameters were introduced. The second DLT occurred in the first patient of the 0.25 mg minnelide and 100 mg/m² protein-bound paclitaxel cohort: a Grade 5 event of sepsis. Five additional patients have been enrolled in the 0.25 mg minnelide and 100 mg/m² protein-bound paclitaxel cohort with no DLTs observed.

Summary of Adverse Events. Overall, the most commonly reported AEs (incidence of >10%) regardless of causality have included: anemia (18%), neutropenia (30%), leukopenia (12%), abdominal distension (12%), abdominal pain (30%), constipation (24%), vomiting (18%), gastroesophageal reflux disease (12%) nausea (24%), Lower extremity oedema (24%), pyrexia (24%), fatigue (18%), chills (12%), local swelling (18%), pain (12%), dehydration (18%), back pain (12%), dizziness (12%), dyspnoea (12%), pleural effusion (12%), skin irritation (12%), and neutrophil count decreased (13%).

### Study Design.

An open-label, Phase 1b study of minnelide given once a day on Days 1 to 5, Days 8 to 12 and Days 15 to 19 in combination with Nab-paclitaxel (Abraxane) plus gemcitabine was administered weekly by IV administration for 3 weeks on Days 1, 8, and 15 as per the Table 1 in the Example. The study was conducted in patients with disease progression while on FOLFIRINOX as first treatment and who have had no prior treatment with nab-paclitaxel [Abraxane] plus gemcitabine or single agent nab-paclitaxel or gemcitabine or in any other combinations. The total number of treatment cycles administered was dependent on drug tolerability by the patient. Approximately 36 patients were enrolled in this study. About 18 patients were enrolled at MTD dose.

Dose-Limiting Toxicity. Dose-limiting toxicities (DLT) were evaluated during Cycle 1 of treatment. Toxicities were graded and documented according to the NCI CTCAE guidelines (briefly defined above). Dose reductions were not allowed during Cycle 1. DLTs include adverse events that are considered to be related to the study drug, including:
Grade 4 neutropenia lasting ≥5 days, Grade 4 neutropenia with use of growth factor, or Grade 3 or 4 neutropenia with fever and/or infection;
Grade 4 thrombocytopenia (or Grade 3 with bleeding);
Grade 3 or 4 treatment-related non-hematological toxicity (Grade 3 nausea, vomiting or diarrhea that last >72 hours despite maximal treatment constitutes a DLT; or a dosing delay of greater than 2 weeks due to a treatment-emergent AEs or related severe laboratory abnormalities.

### Inclusion Criteria for Study Eligibility.

A patient must meet the inclusion criteria listed below:
Signed, written IRB-approved informed consent.
Patients diagnosed with histologically confirmed metastatic adenocarcinoma of the pancreas combined with adenocarcinoma and adenosquamous are allowed, but pure adenosquamous patients are excluded. Tumor progression after receiving standard/approved chemotherapy or where there is no approved therapy.
Patients had disease progression on FOLFIRINOX as first treatment.
Had no prior treatment with nab-paclitaxel (Abraxane) plus gemcitabine or single agent nab-paclitaxel or gemcitabine or in any other combinations.
Patient who has received prior or going to receive any killed vaccine including influenza, pneumococcal or COVID-19 during the study are allowed but any live vaccine like Herpes Zoster is not allowed.
One or more metastatic tumors measurable per on computed tomography (CT) scan per Response Evaluation Criteria in Solid Tumors (RECIST) V1.1 criteria excluding the primary pancreatic lesion.
Karnofsky performance 70% (Cares for self but unable to carry on normal activity or to do active work).
Life expectancy of at least 3 months.
Age ≥19 years.
A negative pregnancy test (if female).
Acceptable liver function:
   Bilirubin≤1.5 times upper limit of normal (ULN).
   Aspartate aminotransferase (AST), serum glutamic oxaloacetic transaminase (SGOT), alanine aminotransferase (ALT), serum glutamic pyruvic transaminase (SGPT), and alkaline phosphatase (ALP)≤2.5×ULN (if liver metastases are present, then ≤5×ULN is allowed).
   Albumin ≥3.0 g/dL.
Acceptable renal function: Serum creatinine within normal limits, OR calculated creatinine clearance ≥60 mL/min/1.73 m² for patients with creatinine levels above institutional normal.
Acceptable hematologic status:
   Baseline absolute neutrophil count (ANC) ≥2000 cells/mm3.
   Platelet count ≥100,000 (plt/mm³).
   Hemoglobin ≥9 g/dL.
Urinalysis: No clinically significant abnormalities.
Acceptable coagulation status:
   Prothrombin time (PT)≤1.5 times institutional ULN.
   Partial thromboplastin time (PTT)≤1.5 times institutional ULN.
   International normalized ratio (INR)≤1.5× institutional ULN.
For men and women of childbearing potential, the use of effective contraceptive methods during the study. From last dose effective contraception is to be maintained as follows:
   Men: 100 days from last dose of minnelide.
   Women of childbearing potential: 6 months from last dose of minnelide.
For females, agree to the use of two physician-approved contraceptive methods (oral, injectable, or implantable hormonal contraceptive; tubal ligation; intra-uterine device; barrier contraceptive with spermicide; or vasectomised partner) while on study IP; and for 3 months following the last dose of IP.
Has negative serum pregnancy test (β-hCG) result at Screening.
For males, must practice true abstinence or agree to use a condom during sexual contact with a pregnant female or a female of childbearing potential while participating in the study, during dose interruptions and for 6 months following IP discontinuation, even if he has undergone a successful vasectomy.

### Dose Interruptions for Minnelide.

A drop in blood counts is usually seen within the first 5-7 days of the start of minnelide and resolves in 4-8 days of withholding of minnelide.

CBC with differential was done on days 1, 8, 15, 22 and more frequently as clinically indicated. Patient were hospitalized for close monitoring when the ANC level was <1000 mm³ (grade 3 or higher) or platelet was <100,000 cells/mm³ at any time. Minnelide™ Capsules would not resume until the ANC level was ≥1500 mm³ or platelet was ≥100,000 cells/mm³.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Treatment Regimen

Minnelide was administered orally once a day on Days 1 to 5, Days 8 to 12 and Days 15 to 19. Nab-paclitaxel (Abraxane) plus gemcitabine was administered weekly by IV administration for 3 weeks on Days 1, 8, and 15 as per the Table 1 dose-escalation schedule. One cycle will be equal to 28 days. Minnelide™ capsules were given with the patient in a fasting state.

At the first dose level 3 patients were treated. If no patients experienced CTCAE Version 5.0 Grade 3 or greater toxicity after a minimum of 3 weeks of treatment, dose escalation proceeded to dose level 2.

If a DLT was observed in 1 out of 3 patients at any dose level, up to an additional 3 patients were enrolled and treated at that dose level. If 2 patients at that dose level had DLTs, dosing was decreased by one-half. If 1 of 6 patients had a DLT, the dose was increased to the second dose level. If 2 or more of the up to 6 patients at the second dose level had DLTs, the preceding dose (dose level 1) was declared the maximum tolerated dose (MTD). If 1 or less of 6 patients had DLT, the dose level 2 was declared the MTD.

TABLE 1
Dose Escalation Regimen.
Dose Escalation

| | Dose Level | | | |
|---|---|---|---|---|
| Dose | Minnelide ™ | Nab-paclitaxel on | Gemcitabine on | Number of |
| Level | Capsules | days 1, 8, and 15 | days 1, 8, and 15 | Patients |
| −1 | 0.25 mg | 75 mg/m² | 600 mg/m² | 3 + 3 |
| 0 | 0.25 mg | 100 mg/m² | 800 mg/m² | 3 + 3 |
| 1 | 0.25 mg | 125 mg/m² | 1000 mg/m² | 3 + 3 |
| 2 | 0.50 mg | 125 mg/m² | 1000 mg/m² | 3 + 3 |
| 3 | 0.75 mg | 125 mg/m² | 1000 mg/m² | 3 + 3 |
| 4 | 1.00 mg | 125 mg/m² | 1000 mg/m² | 3 + 3 |

^{a}Additional dose levels beyond dose level 6 could proceed with minnelide 0.25 mg increments when supported by safety data.

### Example 2. Disease Assessment

Target Lesions. Response criteria for target lesions are shown in Table 2. All measurable lesions up to a maximum of two lesions per organ and five lesions in total, representative of all involved organs, were identified as target lesions and recorded and measured at baseline. Target lesions were selected on the basis of their size (lesions with the longest diameter) and were representative of all involved organs, as well as their suitability for reproducible repeated measurements. All measurements were recorded using calipers if clinically assessed.

A sum of the diameters (longest for non-nodal lesions, short axis for nodal lesions) for all target lesions were calculated and reported as the baseline sum diameters, which were used as a reference to further characterize any objective tumor regression in the measurable dimension of the disease. If lymph nodes were included in the sum, only the short axis contributed.

TABLE 2
Criteria for target lesions.

| | |
|---|---|
| Complete | Disappearance of all target lesions. Any pathological lymph |
| Response | nodes (whether target or non-target) must have reduction in |
| (CR) | short axis to <10 mm. |
| Partial | At least a 30% decrease in the sum of the diameters of |
| Response | target lesions, taking as reference the baseline sum |
| (PR) | of diameters |
| Progressive | At least a 20% increase in the sum of the LD of target |
| Disease | lesions, taking as reference the smallest sum on study (this |
| (PD) | may include the baseline sum). The sum must also |
| | demonstrate an absolute increase of at least 5 mm |
| Stable | Neither sufficient shrinkage to qualify for PR nor sufficient |
| Disease | increase to qualify for PD |
| (SD) | |

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A method for treating pancreatic cancer in a cancer subject, the method which comprises administering to the cancer patient during a 28 day cycle a therapeutically effective combination of:
a) about 0.15 mg to about 2.0 mg of minnelide and/or triptolide according to a first regimen wherein a dose is given once per day on days 1 to 5, 8 to 12, and 15 to 19 of the cycle;
b) about 40 mg/m² to about 125 mg/m² of nab-paclitaxel according to a second regimen wherein a dose is given once per day on days 1, 8, and 15 of the cycle; and
c) about 450 mg/m² to about 1000 mg/m² of gemcitabine according to the second regimen of the cycle;
wherein the cycle is repeated one or more times and the combination effectively treats the pancreatic cancer.

2. The method of claim 1 wherein the cancer subject is administered about 0.15 mg 0.25 mg to about 1.00 mg of minnelide and/or triptolide according to the first regimen.

3. The method of claim 1 wherein the cancer subject is administered about 0.15 mg of minnelide and/or triptolide according to the first regimen, about 40 mg/m² of nab-paclitaxel and about 450 mg/m² of gemcitabine each according to the second regimen.

4. The method of claim 1 wherein the cancer subject is administered about 0.25 mg of minnelide and/or triptolide according to the first regimen, about 100 mg/m² of nab-paclitaxel and about 800 mg/m² of gemcitabine each according to the second regimen.

5. The method of claim 1 wherein the cancer subject is administered about 0.25 mg of minnelide and/or triptolide according to the first regimen, about 125 mg/m² of nab-paclitaxel and about 1000 mg/m² of gemcitabine each according to the second regimen.

6. The method of claim 1 wherein the cancer subject is administered about 0.50 mg of minnelide and/or triptolide according to the first regimen, about 125 mg/m² of nab-paclitaxel and about 1000 mg/m² of gemcitabine each according to the second regimen.

7. The method of claim 1 wherein the cancer subject is administered about 0.75 mg of minnelide and/or triptolide according to the first regimen, about 125 mg/m² of nab-paclitaxel and about 1000 mg/m² of gemcitabine each according to the second regimen.

8. The method of claim 1 wherein the cancer subject is administered about 1.00 mg of minnelide and/or triptolide according to the first regimen, about 125 mg/m² of nab-paclitaxel and about 1000 mg/m² of gemcitabine each according to the second regimen.

9. The method of claim 1 wherein minnelide and/or triptolide is administered orally.

10. The method of claim 1 wherein nab-paclitaxel is administered intravenously, concurrently or sequentially with gemcitabine.

11. The method of claim 1 wherein the subject is further suffering from gastric cancer, ovarian cancer, breast cancer, bladder cancer, skin cancer, or a combination thereof, and the method effectively treats the cancer subject.

12. The method of claim 1 wherein the cancer subject is a human.

13. The method of claim 1 wherein the combination effectively treats the pancreatic cancer without causing a complete blood count of the cancer subject to lower by more than 25% from baseline.

14. The method of claim 1 wherein the combination effectively treats the pancreatic cancer without causing a platelet count or an absolute neutrophil count in the cancer subject to lower by more than 25% from baseline.

15. The method of claim 1 wherein the pancreatic cancer is metastatic adenocarcinoma of the pancreas.
